(19) United States
(12) Patent Application Publication
(10) Pub. No.: US 2025/0146028 A1
(43) Pub. Date: May 8, 2025

(54) AN INTEGRATED PROCESS FOR THE PRODUCTION OF ETHANOL AND PROTEIN FROM RICE DISTILLERY

(71) Applicant: PRAJ INDUSTRIES LIMITED, Pune (IN)

(72) Inventors: Pramod SHANKAR KUMBHAR, Pune (IN); Siddhartha SOURAV PAL, Pune (IN); Sneha NAGNATH PATIL, Pune (IN); Sandip UTTAM NALAWADE, Pune (IN)

(21) Appl. No.: 18/726,335

(22) PCT Filed: Jan. 25, 2023
(86) PCT No.: PCT/IN2023/050080

§ 371 Date: Jul. 2, 2024

(30) Foreign Application Priority Data
Feb. 2, 2022 (IN) 202221005567

(51) Int. Cl. C12P 7/10; C07C 29/80; C12N 9/26; C12N 9/34; C12N 9/42; C12N 9/50; C12P 21/06

(52) U.S. Cl. CPC C12P 7/10; C07C 29/80; C12N 9/2414; C12N 9/2428; C12N 9/2437; C12N 9/50; C12P 21/06; C12Y 302/01001; C12Y 302/01003; C12Y 302/01004

(57) ABSTRACT

The present disclosure relates to an integrated process for obtaining ethanol and proteins from cereal grains (broken rice), the process comprising milling the grains, obtaining a slurry of the milled grains, partially hydrolysing the slurry, saccharifying the partially hydrolysed slurry to obtain saccharified slurry, adding a pre-fermented slurry to the saccharified slurry to obtain ethanol and distilling out the ethanol and separating the remaining as a whole stillage; separating the whole stillage into thin slop and DWG, treating a slurry of the DWG with cellulase enzymes to provide treated DWG slurry, separating the treated DWG slurry into wet cake and filtrate, washing and separating the said wet cake into protein concentrate and a liquid and optionally treating the obtained protein concentrate with alkali or hydrolysing enzymes to obtain purified protein hydrolysate or isolate.

### CROSS-REFERENCE TO RELATED APPLICATIONS AND PRIORITY

The present application claims priority from patent application Ref. no 202221005567 filed on Feb. 2, 2022, incorporated herein by a reference.

### FIELD OF THE INVENTION

The invention relates to the recovery of proteins from the Distiller's Wet Grain (DWG) after fermentation and recovery of ethanol. More particularly, it relates to an integrated process for the recovery of ethanol and proteins from DWG, obtained as a by-product from the distillery.

### BACKGROUND

The global demand for bioethanol has skyrocketed in recent times and traditional alcoholic fermentation has been immensely exploited for this purpose. At present, the global ethanol supply is met mainly by fermenting sugary or starchy grain feedstock. Grains such as rice, wheat, corn, maize, sorghum, rye, barley, oats etc., are used as a readily available source of sugar from starch, or starchy feedstock required for the fermentative process.

Amongst these, grain feedstock such as rice (Oryza sativa L.) is especially valued for its high nutrition and hypoallergenic characteristics. Polished white rice is usually sold as a premium product, while about 14% of the rice is broken into fragments as a by-product from rice mills. As broken rice has the same chemical composition as the premium product and contains a good amount of starch, it serves as an attractive feedstock for ethanol distilleries. Apart from starch, rice also contains a significant quantity of proteins which remains intact even after all starch has been converted to ethanol.

After fermentation and recovery of ethanol, the remaining grain constituents such as protein, lipids, fiber, minerals and vitamins remain relatively unchanged chemically and get concentrated as Distiller's Wet Grain (DWG). Thus, DWG or Distillers' Dried Grains with Soluble (DDGS) are considered valuable by-products of the fermentation of cereal grains. These have a much higher protein content on a dry basis than the original grain and the solid wet cake carries numerous functional proteins and essential amino acids that can be further exploited to generate better functional feed additive(s).

Therefore, isolating or extracting proteins from distillery by-products is desirable for better bioresource utilization, carbon utilization and sustainable management of generated co-products.

A practical and rational strategy is required for economically and effectively utilizing the obtained by-products. Moreover, broken rice, which is plentiful and readily available, may also be used to produce valuable food ingredients. Thus, the present invention relates to an integrated process for the efficient recovery of bioethanol and proteins from cereal grains. The process of the present invention combines carbohydrate and protein pathways to obtain maximum production and recovery of bioethanol and proteins from cereal grains.

Moreover, the proteins recovered from concentrates may be further treated by hydrolytic enzymes or by alkali to furnish purified proteins that are readily commercialized as such or further dried to provide powders having a better shelf-life. Also, the output from the integrated process is enhanced further by integrating additional steps to release more sugars such as by degrading cellulosic structures that could provide more ethanol.

Therefore, the present disclosure relates to an overall high ethanol production from the fermentation of broken rice and further fully utilizing the residual DWG cake comprising proteins for obtaining other valuable products.

### SUMMARY

This summary is not intended to identify all the essential features of the claimed subject matter, nor is it intended for use in determining or limiting the scope of the claimed subject matter. This summary is provided to introduce concepts related to the integrated process for the production of ethanol and protein from the rice distillery, and the concepts are further described below in the detailed description.

The presently disclosed subject matter provides an integrated process for obtaining ethanol and proteins from grains, the process comprising milling the grains, obtaining a slurry of the milled grains, partially hydrolysing the slurry, saccharifying the partially hydrolysed slurry to obtain a saccharified slurry, adding a pre-fermented slurry to the saccharified slurry to obtain ethanol, distilling out the ethanol and separating the remaining as a whole stillage, separating the whole stillage into thin slop and DWG, treating a slurry of the DWG with cellulase enzymes to provide treated DWG slurry, separating the treated DWG slurry into wet cake and filtrate, washing and separating the said wet cake into protein concentrate and a liquid and optionally treating the obtained protein concentrate with alkali, or hydrolysing enzymes to obtain purified proteins.

The said filtrate rich in sugar is used again for slurry preparation after milling the grains or mixed with the saccharified slurry for enhancing ethanol production in the final stream.

In an exemplary embodiment, the instant invention discloses a process for the recovery of hydrolysed proteins from the DWG, arising from a rice distillery wherein the steps include fractionating the broken rice by milling, obtaining the slurry of the milled gains, partially hydrolysing the slurry using liquefying enzymes and saccharifying the partially hydrolysed slurry with glucoamylase to obtain saccharified slurry. The process further comprises steps of separately preparing the pre-fermented slurry to be added to the said saccharified slurry to obtain ethanol, distilling out the ethanol and separating the remaining as a whole stillage. Furthermore, the process comprises the steps of separating the said stillage thin slop and DWG, wherein the slurry of the DWG is treated with cellulase enzymes to provide the treated DWG slurry. The steps further comprise separating the treated DWG slurry into wet cake and filtrate, washing the wet cake and separating into protein concentrate and liquid and optionally treating the obtained protein concentrate with alkali or hydrolysing enzymes to obtain purified proteins.

This summary is not intended to identify all the essential features of the claimed subject matter, nor is it intended to use in determining or limiting the scope of the claimed subject matter.

### BRIEF DESCRIPTION OF DRAWINGS

The detailed description of the drawings is outlined with reference to the accompanying figures. In the figures, the left-most digit(s) of a reference number identifies the Figure in which the reference number first appears. The same numbers are used throughout the drawings to refer like features and components.

FIG. 1: Flowchart depicting the integrated process of fermentation of grains to obtain ethanol and proteins.

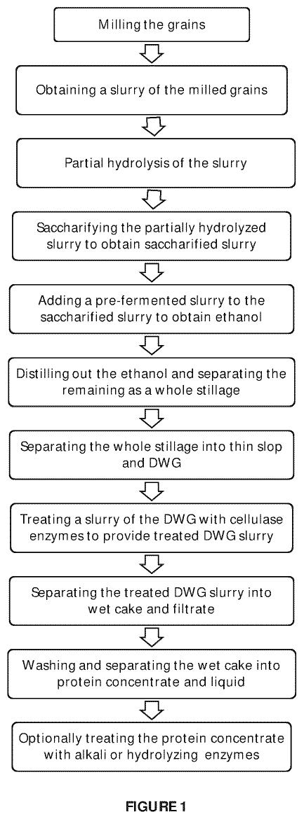

FIG. 2: Flow chart depicting further optional alkali treatment to obtain protein isolate.

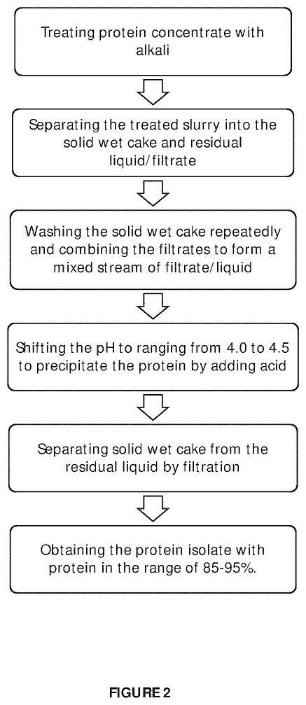

FIG. 3: Flowchart depicting further optional treatment with proteases to obtain protein hydrolysate.

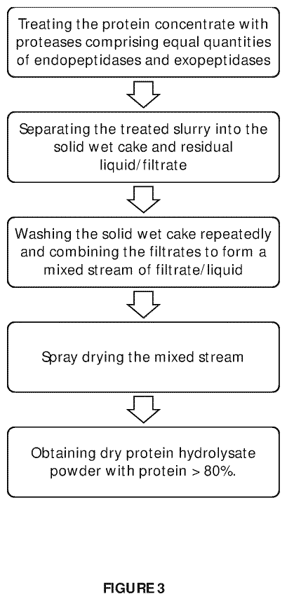

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments.

Before the present process or steps are described, it is to be understood that this disclosure is not limited to any particular process or organism, as there can be multiple possible embodiments that are not expressly illustrated in the present disclosure but may still be practicable within the scope of the present disclosure.

Also, the technical solutions offered by the present disclosure are clearly and completely described below. Examples in which specific reagents or conditions may not have been specified have been conducted under conventional conditions or in a manner recommended by the manufacturer.

### DESCRIPTION

The technical solutions offered by the present disclosure are clearly and completely described below. Examples in which specific compounds or conditions may not have been specified have been conducted under conventional conditions or in a manner recommended by the manufacturer.

In a preferred embodiment, the presently disclosed subject matter provides an integrated process for obtaining ethanol and proteins from grains, the process comprising milling the grains, obtaining a slurry of the milled grains, partially hydrolysing the slurry, saccharifying the partially hydrolysed slurry to obtain a saccharified slurry, adding a pre-fermented slurry to the saccharified slurry to obtain ethanol, distilling out the ethanol and separating the remaining as a whole stillage, separating the whole stillage into thin slop and DWG, treating a slurry of the DWG with cellulase enzymes to provide treated DWG slurry, separating the treated DWG slurry into wet cake and filtrate, washing and separating the said wet cake into protein concentrate and a liquid and optionally treating the obtained protein concentrate with alkali or hydrolysing enzymes to obtain purified proteins

The said filtrate obtained is used again for slurry preparation after milling of grains or mixed with the saccharified slurry for enhancing ethanol production in the final stream.

More particularly, the invention relates to a process for the recovery of proteins from the DWG arising from a rice distillery wherein the steps include fractionating the broken rice by milling, obtaining the slurry of the milled gains, partially hydrolysing the slurry using liquefying enzymes and saccharifying the partially hydrolysed slurry with glucoamylase to obtain saccharified slurry. The process further comprises the steps of separately preparing the pre-fermented slurry to be added to the said saccharified slurry to obtain ethanol, distilling out the ethanol and separating the remaining as a whole stillage. Furthermore, the process comprises the steps of separating the said stillage thin slop and DWG wherein the slurry of the DWG is treated with cellulase enzymes to provide the treated DWG slurry. The steps further comprise separating the treated DWG slurry into wet cake and filtrate, washing the wet cake repeatedly for 2-3 times and separating it into protein concentrate and liquid, said liquid used with first slurry stream to produce ethanol and protein concentrate and optionally treating the obtained protein concentrate with alkali or hydrolysing enzymes to obtain purified proteins.

The term “bioethanol” or “ethanol” is used interchangeably herein with “ethanol” and refers to ethanol generated from the conversion of plant matter.

Rice proteins are widely recognized today for their unique beneficial properties. These proteins provide essential amino acids and are known to control high blood sugar and lower blood pressure and fats. Rice proteins are preferred in vegan diets over whey proteins, which are sourced from animals. Most baby foods also prefer rice protein over milk solids, especially for lactose intolerant infants or as a dietary supplement for people allergic to other common proteins. Furthermore, rice protein-containing products are gaining commercial importance and may serve to provide additional earnings for rice distilleries. Further, it may also address the problem of disposal of spent wash stream, which has a very less shelf life and decomposes rapidly emitting foul odors.

In one embodiment of the present invention, the process includes several steps. Each step has one or more elements for performing the specific function required in an integrated process for obtaining ethanol and proteins from grains. A person skilled in the art may appreciate different variations and/or combinations of these elements that may be used to perform the objects of the invention disclosed herein.

The term “grain” as used herein refers to small, hard, dry fruit (caryopsis) with or without an attached hull layer, harvested for human or animal consumption. There are mainly two types of commercial grain crops, namely cereals and legumes.

In some embodiments, the presently disclosed subject matter provides an integrated process for the production of ethanol and proteins from grains.

Cereal grains such as rice, wheat, corn, maize, sorghum, barley, rye, oats etc. contain starch and protein as major constituents, while the minor constituents include vitamins, phytic acid, lipids, non-starch carbohydrates and minerals. High starch content makes cereals a viable substrate for ethanol production. In a preferred embodiment of the present disclosure, the presently disclosed subject matter provides an integrated process for producing ethanol and protein from grains, the process comprising the grains selected from at least one of rice, wheat, corn, maize, sorghum, rye, barley, oats, or combinations thereof.

Total solids are a measure of the dissolved combined content of all inorganic and organic substances present in a liquid in molecular, ionized, or micro-granular (colloidal sol) suspended form. In an embodiment, the grains comprise starch ranging from 50 to 75%, proteins ranging from 5 to 10%, ash ranging from 0.5 to 1.2%, fats ranging from 0.5 to 5%, and crude fiber ranging from 0.1 to 5%.

To facilitate the hydrolytic release of fermentable sugars, a process of fractionating or breaking down the grains is necessary as to unfold the compact structure and make it amenable for hydrolysis. In an embodiment, the cereals grains are subjected to milling to form a flour with definite particle size to release starch from the substrate. In an embodiment, the particles in the flour are in the range of 0.1 mm to 1.4 mm. In a related preferred embodiment, the grains are milled by mechanical grinding into flour having particle sizes ranging from 0.1 mm to 1.2 mm.

The term “milling” as used herein, refer to breaking down the grains to flour of definite particle size.

The term “slurry”, as used herein, refers to the liquid and solid components of the grain obtained by mixing with water. It is a mixture of denser solid particles suspended in liquid, usually water. In one of the embodiments, a slurry of the milled grains is obtained by mixing with process water. The total solids ranged preferably from 10 to 50% and more preferably in the range of 20 to 35%. In a related preferred embodiment, the slurry of the milled grains comprising total solids ranging from 25 to 30%, obtained by mixing with process water.

During the process of ethanol production, liquefaction and saccharification require the starch granules to be extensively gelatinized. This is an energy-intensive process. Hydrolytic enzymes selectively convert the starch molecules to produce glucose, to be further utilised for the production of ethanol. In some of the embodiments, the hydrolysing enzymes are selected from α-amylase, glucoamylase, glucose isomerase, pullulanase and others. In an embodiment of the present disclosure, partially hydrolysing the slurry is carried out with liquefying enzymes selected from α-amylases.

The term “enzyme” refers to a protein that catalyses the conversion of one molecule into another. The term “enzyme’ as used herein includes any enzyme that can catalyse the transformation of a grain-derived molecule to another grain-derived molecule. Enzymes include those which can degrade or otherwise transform saccharide, cellulose, or lignocellulose molecules to provide fermentable sugars/carbohydrates and/or alcohols.

The terms “hydrolyze” or “hydrolyse” or “hydrolysis” and variations thereof refer to the process of converting polysaccharides (e.g., cellulose) or starch to fermentable sugars, e.g., through the hydrolysis of glycosidic bonds. This process can also be referred to as saccharification. Hydrolysis can be affected with enzymes or chemicals. Hydrolysis products include, for example, fermentable sugars, such as glucose and other small (low molecular weight) monosaccharides, disaccharides, and trisaccharides.

These hydrolytic enzymes depend on factors such as the amorphous or crystalline nature of starch, source of enzymes, substrate and enzyme concentration, temperature, pH and duration etc. In some embodiments, α-amylase is used in the range of 0.30 to 0.60 Kg enzyme/metric ton of starch with enzyme activity 13775 AAU/g and preferably between 0.35 to 0.50 Kg enzyme/metric ton of starch with enzyme activity 13775 AAU/g.

In a related embodiment, the hydrolytic enzymes are used at a pH ranging between 4.0 to 7.0, and preferably between 4.5 to 6.5 at an optimum temperature ranging between 70-100° C. and preferably between 80 to 90° C.

In a related preferred embodiment of the present disclosure, partially hydrolysing of the slurry is carried out with α-amylases ranging from 0.40 to 0.50 Kg enzyme/metric ton of starch with enzyme activity 13775 AAU/g at a pH ranging from 5.0 to 6.0 and at a temperature ranging from 80° C. to 90° C.

Glucoamylase is one of the oldest and widely used biocatalysts in food industry. It causes saccharification of partially processed starch/dextrin to glucose, which is an essential substrate for fermentation processes. These are the exo-acting enzymes that tend to release consecutive glucose units from the non-reducing ends of the starch molecules. In an embodiment of the present disclosure, saccharifying the partially hydrolysed slurry is carried out with glucoamylase.

In one embodiment of the present disclosure, the glucoamylase is used preferably in the range of 1.0 to 2.0 Kg enzyme/ton of starch with enzyme activity 380 GAU/g at an optimum temperature of 20 to 40° C. and preferably 25 to 35° C. In a related preferred embodiment, saccharifying the partially hydrolysed slurry is carried out with glucoamylase ranging from 0.7 to 1.0 Kg/ton with enzyme activity 380 GAU/g of starch at a temperature of 32° C. to obtain a completely hydrolysed slurry.

Simultaneous hydrolysis, saccharification and fermentation process has been introduced to increase ethanol yield and to save energy and investment cost. The enzymes such as α-amylase, glucoamylase are added to the slurry, concomitantly with yeasts. The process is conducted at an ambient temperature for a definite duration.

“Fermentation” as used herein refers to the breaking down of sugar molecules into simpler compounds, to produce substances that can be used in making chemical energy. In some of the embodiments, the process of fermentation results in the formation of ethanol and separating the remaining as a whole stillage.

The presence of yeast along with enzymes minimizes sugar accumulation. Since the sugar is produced slowly during the starch breakdown, higher yields and concentrations of ethanol are possible. In an embodiment, the pre-fermented slurry is prepared separately and the said slurry comprising liquefied slurry and the S. cerevisiae in the range of 0.10 to 0.40 Kg/KL and preferably in the range of 0.15 to 0.35 Kg/KL.

In one embodiment, the saccharified slurry comprised glucoamylase preferably in the range of 0.10 to 0.20 Kg/MT starch with enzyme activity 380 GAU/g.

In a further embodiment, the saccharified slurry comprised of urea in the range of 100 to 1000 ppm and more preferably 250 to 750 ppm.

The temperature control in combination with enzymatic hydrolysis using starch hydrolysing enzymes could significantly improve the efficiency of the fermentation process.

In one embodiment, the process of fermentation is carried out at an optimum temperature in the range of 20 to 40° C. and preferably in the range between 25 to 35° C. Further, fermentation is carried out for 30 to 70 hrs and preferably for 40 to 65 hours.

In a preferred embodiment of the present disclosure, the pre-fermented slurry is prepared separately and added to the saccharified slurry, the said slurry comprising liquefied slurry, S. cerevisiae in the range of 0.20 to 0.30 Kg/KL, glucoamylase in the range of 0.15 Kg/MT starch with enzyme activity 380 GAU/g and urea at about 500 ppm to obtain ethanol by fermentation, wherein the fermentation is carried out at a temperature ranging from 32 to 34° C. for 48 to 60 hours using S. cerevisiae.

After fermentation, -ethanol is distilled out and collected in a container. In an embodiment of the instant disclosure, the ethanol obtained was at least 90% pure. In a further related embodiment, separating the whole stillage from the obtained ethanol after distillation provides ethanol of at least 99% purity.

In some of the embodiments, the distillation separates the ethanol from the fermented wash.

The process of fermentation produces a by-product called Dried Distiller's Grain with solubles (DDGS) or Distiller's Wet Grain (DWG). Since only starch and sugars are converted into ethanol, nonfermentable components in cereal grains are concentrated in DDGS or DWG. Currently, most of these by-products have been used as an ingredient for livestock feed. These contain the fiber, fats, protein, other unfermented components of the grain and yeast cells.

In one of the embodiments of the present invention of an integrated process of obtaining ethanol and proteins from grains separating the whole stillage into thin slop and DWG, is by decantation. In a further related embodiment, the DWG comprising crude proteins in the range of 60 to 65% is mixed with water to obtain DWG slurry.

Commercially available cellulases are used to convert celluloses to produce glucose. In another embodiment, the cellulases are preferably in the range of 0.05 to 2% at an optimum pH range preferably between 4.0 to 6.0.

“Cellulase’ is used herein generally to refer to enzymes involved in degradation of cellulosic material.

Further in a related embodiment, treating the slurry of the DWG with cellulase enzymes comprises the steps of treating the DWG slurry with cellulases in the range of 0.1 to 1% on a dry basis at a pH ranging from 4.5 to 5.5, to obtain solid protein wet cake and remaining residual liquid, separating the solid wet cake from the residual liquid by filtration and washing the solid wet cake repeatedly with an equal quantity of water to obtain the protein concentrate with protein ranging from 80 to 85%.

“Protein concentrate” used herein refers to the product made by removal of sufficient non-protein components from rice so that the protein concentration ranges between 60-85% w/w.

It is well known that the degree of protein solubility in an aqueous medium is the result of electrostatic and hydrophobic interaction between protein molecules and proteins. Proteins are extracted when electrostatic repulsion between proteins is greater than hydrophobic interactions. The proteins are found to show increased solubility at extreme pH of 2.0 to 12.0. Further, the high viscosity is mainly due to the expansion and partial solvation of protein aggregates. The pH of the dissolved protein solution is adjusted to the isoelectric point and as result, the proteins are precipitated by adjusting the pH of the solution or pH shift to the isoelectric point. The precipitated proteins are then recovered.

In an embodiment of the present disclosure, the alkali is preferably used in the range of 0.05 to 4.0M, at a temperature in the range of 20 to 70° C., with a pH ranging between 10.0 to 14.0. The shift in the pH is carried out using various acids such as HCl, H₂SO₄, HNO₃, CH₃COOH. The shift in the pH for the protein precipitation preferably ranged between 3.0 to 5.5.

In a related embodiment, treating the protein concentrate with alkali comprises the steps of treating protein concentrate with the alkali, in the range of 0.1 to 2.0M at a temperature ranging from 30 to 60° C. and at a pH ranging from 10.5 to 12.5, separating the treated slurry into the solid wet cake and residual liquid/filtrate, washing the solid wet cake repeatedly and combining the filtrates to form a mixed stream of filtrate/liquid, shifting the pH to ranging from 4.0 to 4.5 to precipitate the protein by adding acid and separating solid wet cake from the residual liquid by filtration to obtain the protein isolate with protein in the range of 85-95%.

The terms “protein isolate” refers to a refined form of proteins, which undergoes further processing after concentrate and is obtained in further purified form.

The term “proteases” refer to the enzymes that catalyse the proteolysis or breaking down of proteins into smaller polypeptides, or single amino acids by cleaving the peptide bonds within the proteins by hydrolysis. Proteases are subdivided into two major groups, i.e., exopeptidases and endopeptidases, depending on their site of action. Exopeptidases cleave the peptide bond proximal to the amino or carboxy termini of the substrate, whereas endopeptidases cleave peptide bonds distant from the termini of the substrate.

In one embodiment, the proteases are used at an optimum temperature between 40 to 70° C. and more preferably between 45 to 65° C. In another embodiment, the pH required for the process ranged between 3.0 to 7.0 and preferably at an optimum pH of 4.0 to 6.0.

In a further preferred embodiment of the present disclosure of an integrated process for the production of ethanol and protein comprises the steps of treating the protein concentrate with proteases comprising equal quantities of endopeptidases and exopeptidases at a temperature ranging from 50 to 60° C. and a pH ranging from 4.5 to 5.5, separating the treated slurry into the solid wet cake and residual liquid/filtrate, washing the solid wet cake repeatedly and combining the filtrates to form a mixed stream of filtrate/liquid and spray drying the mixed stream at outlet temperature ranging from 80° C. to 100° C. and input temperature ranging from 180° C. to 190° C. to obtain dry protein hydrolysate powder with protein >80%.

“Protein hydrolysate” refers to the mixtures of polypeptides, oligopeptides and amino acids, as produced from the partial hydrolysis of intact proteins having protein concentration ranging from 75-85% w/w.

Various modifications to the embodiments will be readily apparent to those skilled in the art and the generic principles herein may be applied to other embodiments. However, one of ordinary skill in the art will readily recognize that the present disclosure is not intended to be limited to the embodiments illustrated but is to be accorded the widest scope consistent with the principles and features described herein.

The foregoing description shall be interpreted as illustrative and not limiting in any sense. A person of ordinary skill in the art would understand that certain modifications could come within the scope of this disclosure.

The features and properties of the present disclosure are described in further detail below with reference to examples.

### EXAMPLES

The following examples have been included to provide illustrations of the presently disclosed subject matter. Considering the present disclosure and the general level of skill in the art, those of skill will appreciate that the following examples are intended to be exemplary only and that numerous changes, modifications and alterations can be employed without departing from the spirit and scope of the presently disclosed subject matter.

### Source of Material:

Broken rice is obtained from India, the enzymes are commercially available and yeast is procured from China.

### Example 1: Broken Rice to Ethanol and Protein

A batch of about 3.75 kg of broken rice with total solids about 90% by weight and starch about 70% w/w, proteins about 8% w/w, ash about 1% w/w, fats about 1.2% w/w, crude fibers about 0.3% w/w was used as feedstock. It was subjected to mechanical grinding by dry milling to get 20% to 25% of the rice flour having particle size of about 0.3 mm, 60%-70% of about 0.3 mm to 0.8 mm, 8% to 10% of about 0.8 mm to 1.0 mm, 2% to 6% of about 1.0 mm to 1.2 mm and none above 1.2 mm. To the flour obtained, 7.72 kg of process water was added to make the slurry of 11.47 kg with 30% total solids. The pH of the slurry was adjusted to 5.5 using 1 ml sulfuric acid. Further, to the said slurry 1.18 g of starch liquefying α-amylase enzyme with enzyme activity 13775 AAU/g, considering that a dose of 0.45 kg/metric ton of starch was added. Liquefaction was carried out at 88° C. for 3 hours. As a result, the starch was partially hydrolysed converting starch to dextrin. The liquefied slurry obtained was cooled down to 32° C. and 2.2 g of saccharifying glucoamylase enzyme with enzyme activity 380 GAU/g was added considering dose 0.85 kg/metric ton of starch along with PF quantity of 2.3 kg (1.72 g S. cerevisiae, 1 kg/Kl dose). Glucoamylase hydrolyses partially hydrolysed starch from liquefaction to yield glucose units and the yeast ferments these glucose units to ethanol. Fermentation was carried out at 32° C. with a reaction time of 57 hours. The ethanol formed was 1.71 L from batch of 13.77 kg.

After fermentation, the fermented wash was subjected to distillation to distill out 1.71 L of ethanol with >99% purity. Post-distillation, the spent wash was subjected to solid-liquid separation using a centrifuge. The solid cake (DWG) obtained post-filtration was 2.5 kg with 30% total solids and thin slop having 9.14 kg with total solids of 3.5% w/w. This DWG is crude protein.

Compositional analysis of the said DWG exhibited proteins ranging from 60 to 70% w/w with starch in the range of 0.5 to 0.7% w/w (Table 1).

TABLE 1
Composition of Rice-Based DWG

| Sl. No | Components (% w/w) | Values |
|---|---|---|
| 1 | Protein | 63 |
| 2 | Glucan | 4 |
| 3 | Xylan | 2.5 |
| 4 | Starch | 0.6 |
| 5 | Fats | 3.1 |
| 6 | Ash | 3 |

### Example 2: Cellulase Treatment

About 2.5 kg DWG was mixed with 2.5 kg fresh water to make slurry of 5 kg with 15% total solids. The pH of the slurry was adjusted to 5.2 using 15 ml 20% w/w NaOH solution. 1.4 g of cellulase enzyme was added to the said slurry considering an enzyme dose of 30 mg/g of cellulose. Fiber hydrolysis was carried out for 24 hours at 55° C. This slurry was subjected to solid-liquid separation using a centrifugal filtration carried out at 1400 rpm resulting in 2.3 kg of solid cake and 2.7 kg of filtrate. 2.3 kg solid cake was washed again using 2.3 kg fresh water. The obtained slurry was subjected to solid-liquid separation by centrifugal filtration at 1400 rpm resulting in about 2.1 kg of solid cake. Filtrates obtained were pooled and used for further ethanol production. The obtained cake was dried at 40° C. for 24 hours. 0.7 kg of protein concentrate was obtained with 90% solids by weight. The composition of the resulting cake containing proteins was analysed, wherein the protein concentrate contained about 82% protein by weight (Table 2).

TABLE 2
Compositional analysis of Protein concentrate sample

| Sl. No | Components (% w/w) | Values |
|---|---|---|
| 1 | Moisture | 5 |
| 2 | Protein | 82 |
| 3 | Fats | 0.8 |
| 4 | Ash | 1.4 |
| 5 | Carbohydrates and Fibers | 3 |
| | Heavy Metals (ppm) | |
| 6 | Lead | 0.15-0.3 |
| 7 | Mercury | Not detected |
| 8 | Arsenic | Not detected |

Thus, the DWG obtained after distillation from whole stillage provided the protein concentrates as a value-added product. Further, the obtained DWG may be alternately processed by various other methods such as treatment with alkali or proteases to furnish protein isolates and protein hydrolysate respectively.

### Example 3: Protein Isolate from Protein Concentrate

About 0.38 kg of protein concentrate was mixed with 4.62 kg of 0.25M NaOH solution to obtain a slurry of 5 kg with 7% solids by weight, wherein 46.2 grams of NaOH was added to 4.57 kg of fresh water to make 0.25M NaOH solution. This slurry was subjected to alkaline treatment for 3 hours at 60° C. The said slurry was cooled down to room temperature and subjected to solid-liquid separation using a centrifuge. 4.37 kg of the filtrate was obtained with isolated protein and was called Filtrate 1. The solid cake obtained was 0.63 kg. This cake was washed with 0.63 kg of fresh water to recover leftover protein in the cake. This slurry was subjected to solid-liquid separation. The filtrate obtained was 0.84 kg and was called Filtrate 2. The cake obtained was 0.42 kg. Filtrate 1 and Filtrate 2 were pooled, and the total final filtrate obtained was 5.21 kg having pH of 12.3. This filtrate was further precipitated using 1N HCl solution at pH 4.3 to produce the isolated protein. This precipitate was repeatedly washed with fresh water to obtain purified isolated protein. About 0.547 kg of precipitate was obtained. This precipitate was dried at 40-50° C. to obtain about 0.248 kg rice protein having 90% w/w protein with 95% purity (Table 3).

TABLE 3
Compositional analysis of Protein Isolate

| Sl. No | Components (% w/w) | Values |
|---|---|---|
| 1 | Moisture | 5 |
| 2 | Protein | 90 |
| 3 | Fats | 1.3 |
| 4 | Ash | 1.4 |
| | Heavy Metals (ppm) | |
| 5 | Lead | 0.15-0.3 |
| 6 | Mercury | Not detected |
| 7 | Arsenic | Not detected |

### Example 4: Treatment with Alkali

The process of Example 3 was repeated using KOH and NaOH under the same experimental conditions but with different alkaline dosages. Results are shown in Table 4.

The protein recovered after pH shift at the isoelectric point ranged between 65-75% and 65 to 80% (Table 4) for KOH and NaOH respectively.

TABLE 4
Extraction of protein isolate using KOH and NaOH

| Wet Extraction | | | | pH shift | | |
|---|---|---|---|---|---|---|
| Wet chemical | | Protein | pH after wet | pH shift | Dosage | Protein |
| extraction | Dosage (M) | recovery | extraction | conditions | HCl (N) | recovery (%) |
| KOH | 0.1 | 15-20% | 11-11.5 | 4-4.5 | 1 | 7-10 |
| | 0.5 | 65-70% | 12-12.5 | 4-4.5 | 1 | 37-45 |
| | 1 | >85% | 12-12.5 | 4-4.5 | 1 | 65-75 |
| | 1.5 | >85% | 12-12.5 | 4-4.5 | 1 | 65-75 |
| | 2 | >85% | 12-12.5 | 4-4.5 | 1 | 65-75 |
| NaOH | 0.1 | 20-25 | 11-11.5 | 4-4.5 | 1 | 10-15 |
| | 0.5 | 65-70 | 12-12.5 | 4-4.5 | 1 | 40-45 |
| | 1 | 80-85 | 12-12.5 | 4-4.5 | 1 | 65-75 |
| | 1.5 | 85-90 | 12-12.5 | 4-4.5 | 1 | 65-75 |
| | 2 | 85-92 | 12-12.5 | 4-4.5 | 1 | 75-80 |

### Example 5: Protein Hydrolysate from Protein Concentrate

The protein concentrate obtained was used further for protein extraction and hydrolysis. A batch of 5 kg was conducted to extract and hydrolyze the protein. 0.83 kg protein concentrate was mixed with 4.17 kg fresh water to make a slurry of 5 kg with 15% total solids. The pH of the slurry was adjusted to 5.2 using 15 ml 20% w/w NaOH solution. To the said slurry, 11.25 g of endopeptidase and 14.94 g of exopeptidase (1.5% of the total slurry solids) were added to facilitate enzyme hydrolysis. The slurry was subjected to protease treatment at 55° C. for a retention time of 48 hours at 200 rpm.

In the next step, the hydrolyzed slurry was subjected to filtration for solid-liquid separation. Centrifugal filtration was carried out at 1400 RPM for 30 minutes at room temperature. About 1.7 kg of solid cake was obtained and 3.3 kg of filtrate was obtained. This filtrate was called Filtrate 1. The solid cake was further washed by adding 1.7 kg of fresh water and passed through centrifugal filtration at 1400 rpm for 30 minutes at room temperature. 2 kg of the filtrate was obtained. This filtrate was called Filtrate 2. Filtrate 1 had 7.16% protein with 7.89% total solids and Filtrate 2 had 2.41% protein with 2.71% total solids. Filtrate 1 and Filtrate 2 were pooled and was called Filtrate 3. Filtrate 3 had 5.36% protein and 5.93% total solids. This filtrate was then spray dried at outlet temperature ranging from 80 to 100° C. and input temperature of 180-190° C. The total hydrolyzed protein product obtained was 330 g of protein powder was obtained with 90% total solids and 85% purity and 32% Degree of Hydrolysis (Table 5). The amino acid profiling was carried out for the protein hydrolysate (Table 6).

TABLE 5
Compositional analysis of hydrolyzed protein

| S. No | Components(% w/w) | Values |
|---|---|---|
| 1 | Moisture | 10 |
| 2 | Proteins | 85 |
| 3 | Fats | |
| 4 | Ash | 0.8 |
| 5 | Carbohydrates and Fibers | 10 |
| | Heavy Metals (ppm) | |
| 6 | Lead | 0.15-0.3 |
| 7 | Mercury, Arsenic | Not detected |
| | Aflatoxins (μg/Kg) | |
| 9 | Aflatoxin G2, B2, B1, Ocharotoxin | Below detection limit |
| 10 | Aflatoxin G2 | Below detection limit |
| | Microbiology report (cfu/g) | |
| 14 | Total plate count | 4727 |
| 15 | Yeast and mould | 2000 |
| 16 | E. Coli | absent |
| 17 | Salmonella | Not detected |
| 18 | Coliferms | <10 |

TABLE 6
Amino acids profile of protein hydrolysate

| S. No | Amino Acid Profile (% w/w) | Rice Hydrolyzed Protein |
|---|---|---|
| 1 | Aspartic Acid | 70485 |
| 2 | Glutamic Acid | 139331.78 |
| 3 | Serine | 33418.02 |
| 4 | Histadine | 23077.55 |
| 5 | Glycine | 30113.79 |
| 6 | Threonine | 25625.17 |
| 7 | Arganine | 32828.32 |
| 8 | Alanine | 77449.7 |
| 9 | Tyrosine | 31231 |
| 10 | Cystine | 1849.54 |
| 11 | Valine | 43925.41 |
| 12 | Methionine | 11929.64 |
| 13 | Phenyl Alanine | 35880.4 |
| 14 | Isoleucine | 30230.77 |
| 15 | Leucine | 48358.09 |
| 16 | Lysine | 31392.34 |
| 17 | Hydroxy proline | 32943.19 |
| 18 | Proline | 49595.93 |
| | Total | 749665.64 |

We claim:

1. An integrated process for obtaining ethanol and proteins from grains comprising:
a) milling the grains;
b) obtaining a slurry of the milled grains;
c) partially hydrolysing the slurry;
d) saccharifying the partially hydrolysed slurry to obtain saccharified slurry;
e) adding a pre-fermented slurry to the saccharified slurry to obtain ethanol;
f) distilling out the ethanol and separating the remaining as a whole stillage;
g) separating the whole stillage into thin slop and DWG;
h) treating a slurry of the DWG with cellulase enzymes to provide treated DWG slurry;
i) separating the treated DWG slurry into wet cake and filtrate;
j) washing and separating the wet cake into protein concentrate and liquid; and
k) optionally treating the protein concentrate with alkali or hydrolysing enzymes to obtain purified proteins.

2. The process as claimed in claim 1, wherein the grains are selected from at least one of rice, wheat, corn, maize, sorghum, rye, barley, oats or combinations thereof.

3. The process as claimed in claim 1, wherein total solids in grains comprise starch ranging from 50 to 75%, proteins ranging from 5 to 10%, ash ranging from 0.5 to 1.2%, fats ranging from 0.5 to 5% and crude fiber ranging from 0.1 to 5%.

4. The process as claimed in claim 1, wherein the grains are milled by mechanical grinding into flour having particle sizes ranging from 0.1 mm to 1.2 mm.

5. The process as claimed in claim 1, wherein the slurry of the milled grains comprising total solids ranging from 25 to 30% is obtained by mixing with process water.

6. The process as claimed in claim 1, wherein partially hydrolysing the slurry is carried out with α-amylases ranging from 0.40 to 0.50 Kg enzyme/metric ton of starch with enzyme activity 13775 AAU/g at a pH ranging from 5.0 to 6.0 and at a temperature ranging from 80° C. to 90° C.

7. The process as claimed in claim 1, wherein saccharifying the partially hydrolysed slurry is carried out with glucoamylase ranging from 0.7 to 1.0 Kg/ton with enzyme activity 380 GAU/g of starch at a temperature ranging from 32° C. to obtain a completely hydrolysed slurry.

8. The process as claimed in claim 1, wherein the pre-fermented slurry is prepared separately and added to the saccharified slurry; the said slurry comprising liquefied slurry, S. cerevisiae in the range of 0.20 to 0.30 Kg/KL, glucoamylase in the range of 0.15 Kg/MT starch with enzyme activity 380 GAU/g and urea at about 500 ppm to obtain ethanol by fermentation, wherein the fermentation is carried out at a temperature ranging from 32 to 34° C. for 48 to 60 hours using S. cerevisiae.

9. The process as claimed in claim 1, wherein separating the whole stillage from the obtained ethanol after distillation provides ethanol of at least 99% purity.

10. The process as claimed in claim 1, wherein separating the whole stillage into thin slop and DWG is by decantation.

11. The process as claimed in claim 11, wherein the DWG comprising crude proteins in the range of 60 to 65% is mixed with water to obtain DWG slurry.

12. The process as claimed in claim 1, wherein treating the slurry of the DWG with cellulase enzymes comprises the steps of:
a) treating the DWG slurry with cellulases in the range of 0.1 to 1% on a dry basis at a pH ranging from 4.5 to 5.5 to obtain solid protein wet cake and remaining residual liquid;
b) separating the solid wet cake from the residual liquid by filtration
c) washing the solid wet cake repeatedly with an equal quantity of water
to obtain the protein concentrate with protein ranging from 80 to 85%.

13. The process as claimed in claim 1, wherein treating the protein concentrate with alkali comprises the steps of:
a) treating protein concentrate with the alkali, in the range of 0.1 to 2.0M at a temperature ranging from 30 to 60° C. and at a pH ranging from 10.5 to 12.5;
b) separating the treated slurry into the solid wet cake and residual liquid/filtrate;
c) washing the solid wet cake repeatedly and combining the filtrates to form a mixed stream of filtrate/liquid;
d) shifting the pH to ranging from 4.0 to 4.5 to precipitate the protein by adding acid;
e) separating solid wet cake from the residual liquid by filtration;
to obtain the protein isolate with protein in the range of 85-95%.

14. The process as claimed in claim 1, wherein treating the protein concentrate with the proteases comprises the steps of:
a) treating the protein concentrate with proteases comprising equal quantities of endopeptidases and exopeptidases at a temperature ranging from 50 to 60° C. and a pH ranging from 4.5 to 5.5;
b) separating the treated slurry into the solid wet cake and residual liquid/filtrate;
c) washing the solid wet cake repeatedly and combining the filtrates to form a mixed stream of filtrate/liquid;
d) spray drying the mixed stream at outlet temperature ranging from 80° C. 100° C. and input temperature ranging from 180° C. to 190° C.;
to obtain dry protein hydrolysate powder with protein >80%.
